(19) United States
(12) Patent Application Publication
(10) Pub. No.: US 2008/0160117 A1
(43) Pub. Date: Jul. 3, 2008

(54) COMPOSITION, A FOOD, AND A BEVERAGE HAVING AN ANTI-OBESITY ACTION

(75) Inventors: Naoto KOYAMA, Kawasaki-shi (JP); Hideki Mori, Kawasaki-shi (JP); Yuki Okabe, Kawasaki-shi (JP); Hiroyuki Tanimoto, Kawasaki-shi (JP)

Correspondence Address: OBLON, SPIVAK, MCCLELLAND MAIER & NEUSTADT, P.C., 1940 DUKE STREET, ALEXANDRIA, VA, 22314, US

(73) Assignee: Ajinomoto Co., Inc., Tokyo (JP)

(21) Appl. No.: 12/043,573

(22) Filed: Mar. 6, 2008

Related U.S. Application Data
(62) Division of application No. 11/226,246, filed on Sep. 15, 2005.

(30) Foreign Application Priority Data
Sep. 29, 2004 (JP) 2004-283745

(51) Int. Cl. A61K 36/48; A61P 3/04

(52) U.S. Cl. 424/757

(57) ABSTRACT

Compositions, foods, and beverages which contain a salt-free or a reduced-salt fermented soybean paste-like food material in which the salt content is 5% or less, obtained by hydrolyzing soybean by a koji mold have an excellent anti-obesity action.

### CROSS REFERENCES TO RELATED APPLICATIONS

This application claims priority to Japanese Patent Application No. 283745/2004, filed on Sep. 29, 2004, and which is incorporated herein by reference in its entirety.

### BACKGROUND OF THE INVENTION

1. Field of the Invention

The present invention relates to compositions, foods, and beverages which have an anti-obesity action. The present invention also relates to methods of treating or preventing obesity, i.e., controlling weight gain or effecting weight loss, by ingestion of such a composition, food, or beverage.

2. Discussion of the Background

Recently, the chances to take animal proteins and lipids such as dairy products, meats, and eggs have increased voluminously along with the Westernization of the eating habits in Japan. As a result, the ratio of obese persons is increasing while the gourmet aspects of food and the development of the food service industry advance. Obesity is a type of lifestyle disease that is referred to as the deadly quartet along with high blood pressure, hyperlipemia, and diabetes, and it becomes a further, risk factor for diseases such as arteriosclerosis. Therefore, to prevent obesity, therapeutic exercise and an improvement of eating habits, etc. are advocated.

In this social background, studies on foods having anti-obesity actions to prevent or cure obesity have been carried out. For example, as a dietary fiber, the use of polygalactomannan or indigestible dextrin has been reported. Polygalactomannan gives a person a sensation of fullness for a longer period of time since it stays in the stomach for a long period of time (see, Krotkiewski M., Br. J. Nutr., vol. 52, p. 97 (1984)). Indigestible dextrin has a low amount of calories (1.3-1.5 kcal/g) (see, Ohkuma et al, New Food Industry, vol. 35(10), pp. 47-52 (1993)).

In addition, oils which are not accumulated easily due to differences in the structure or the fatty acid composition, such as diacylglycerol (see, Nagao, T. et al, J. Nutr., vol. 130(4), pp. 792-7 (2000)) and medium chain fatty acid triacylglycerol (see, Tsuji, H. et al, J. Nutr., vol. 131(11), pp. 2853-9 (2001)) or substances having effects on burning body fat, such as capsaicin (see, Kawada et al, J. Nutr., vol. 116, pp. 1272-1278 (1986), have been reported.

Recently, traditional Japanese foods have been reviewed. Especially, soybean which is abundant in high quality plant protein is the raw material of various fermented foods such as fermented soybean paste (miso), soy sauce, fermented soybeans (natto), and so on. Due to its high nutritive value, soybean has attracted attention. Needless to say, men and women of all ages can take it daily and safely since it has been taken for years.

Recently, the physiological action of the soybean has begun to attract attention. In regard to the anti-obesity actions of soybean materials, the action of soybean peptides to reduce weight of mice (see, Saitou, M., Nutr. Sci. Soy Protein, Jpn., vol. 12, pp. 91-94 (1991)), the action of soybean peptides to reduce body fats of mice (see, Niiho, Y., Yakugaku zasshi. Journal of the Pharmaceutical Society of Japan, vol. 113(4), pp. 334-392 (1993)), and the action of soybean peptides to reduce body fats of humans (see, Komatsu, T., Nutr. Sci. Soy Protein, Jpn., vol. 11. pp. 98-103 (1990)) have been reported.

However, soybean peptides are not suitable for wide application due to their characteristic bitterness even though they are interesting materials. Moreover, no studies on an anti-obesity action of soybean hydrolyzed to amino acids like fermented soybean paste (miso) have been reported.

### SUMMARY OF THE INVENTION

Accordingly, it is one object of the present invention to provide novel compositions, foods, and beverages which have an anti-obesity action.

It is another object of the present invention to provide novel methods of treating or preventing obesity in humans and animals.

It is another object of the present invention to provide novel methods for controlling weight gain in humans and animals.

It is another object of the present invention to provide novel methods for effecting weight loss in humans and animals.

These and other objects, which will become apparent during the following detailed description, have been achieved by the inventors' discovery that a composition, a food, or a beverage which contains a salt-free or a reduced-salt fermented soybean paste-like food material obtained by hydrolyzing soybean in a salt-free or low-salt condition has an excellent anti-obesity action.

Accordingly, the invention provides the following:

(1) A salt-free or a reduced-salt fermented soybean paste-like food material having an anti-obesity action, obtained by hydrolyzing soybean or a mixture of soybean and a sub-material with a koji mold, wherein the salt content in the fermented soybean paste-like food material is 5% by weight, based on the total weight of the fermented soybean paste-like food material, or less.

(2) A food material according to (1), wherein said sub-material is rice and/or wheat.

(3) A food material according to (1), wherein said Koji mold is A. sojae and/or A. oryzae.

(4) A food material according to (1), wherein said fermented soybean paste-like food material is prepared by a method comprising:

(i) adding a koji mold and a culture broth of a bacteriocin producing lactic acid bacterium, or a supernatant thereof, to soybean or a mixture of soybean and a sub-material:

(ii) making koji in a koji-making machine in which the outside air is excluded, with the exception of a time period of “TEIRE”-treatment, while supplying sterilized air continuously or intermittently;

(iii) mixing the resulting koji with a culture broth of a bacteriocin producing lactic acid bacterium, or a supernatant thereof, to form a koji and culture broth mixture, if necessary, additionally mixing the koji and culture broth mixture with a food material selected from the group consisting of rice, wheat, soybean, and mixtures thereof in an amount of 0.01 to 50 times by weight of the resulting koji to form a koji, culture broth, and food material mixture;

(iv) forming an unrefined soy by making the paste form of the koji, culture broth and food material mixture; and

(v) hydrolyzing the unrefined soy in a low salt condition to obtain said fermented soybean paste-like food material in which the salt content is 5% by weight, based on the total weight of the fermented soybean paste-like food material, or less.

(5) A food material according to (4), wherein said sub-material is at least one member selected from the group consisting of rice, wheat, and mixtures thereof.

(6) A food material according to (4), wherein said bacteriocin is nisin.

(7) A food material according to (4), wherein the nisin content in the culture broth of a lactic acid bacterium or the supernatant thereof is 1000 IU/ml or more.

(8) A food material according to (4), wherein the lactic acid bacterium is Lactococcus lactis.

(9) A food material according to (1), wherein said food material is contained in a package and an indication that said food material has an anti-obesity action is labeled on the package.

(10) A food or a beverage, which comprises a salt free or a reduced salt fermented soybean paste-like food material having an anti-obesity action obtained by hydrolyzing soybean or a mixture of soybean and a sub-material by a koji mold, and wherein the salt content in the fermented soybean paste-like food material is 5% by weight, based on the total weight of the fermented soybean paste-like food material, or less.

(11) A food or a beverage according to (10), wherein said fermented soybean paste-like food material is prepared by steps comprising:

(i) adding a koji mold and a culture broth of a bacteriocin producing lactic acid bacterium, or a supernatant thereof, to soybean or a mixture of soybean and a sub-material;

(ii) making koji in a koji-making machine in which the outside air is excluded, with the exception of a time period of “TEIRE”-treatment, while supplying sterilized air continuously or intermittently;

(iii) mixing the resulting koji with a culture broth of a bacteriocin producing lactic acid bacterium, or a supernatant thereof, to form a koji and culture broth mixture, if necessary, additionally mixing the koji and culture broth mixture with a food material selected from the group consisting of rice, wheat, soybean, and mixtures thereof in an amount of 0.01 to 50 times by weight of the resulting koji to form a koji, culture broth, and food material mixture;

(iv) forming an unrefined soy by making the paste form of the koji, culture broth and food material mixture; and

(v) hydrolyzing the unrefined soy in a low salt condition to obtain said fermented soybean paste-like food material in which the salt content is 5% by weight, based on the total weight of the fermented soybean paste-like food material, or less.

(12) A food or a beverage according to (10), wherein said food and beverage is a seasoning, a soup, a sauce, a dressing, jam, a cereal, bread, confectionery, a luxury beverage, or a cooked processed food.

(13) A food or a beverage according to (10), wherein said food or beverage is contained in a package and an indication that said food or beverage has an anti-obesity action is labeled on the package.

(14) A method of treating or preventing obesity, comprising administering to a subject in need thereof an effective amount of a food material according to (1).

(15) A method of controlling weight gain, comprising administering to a subject in need thereof an effective amount of a food material according to (1).

(16) A method of effecting weight loss, comprising administering to a subject in need thereof an effective amount of a food material according to (1).

(17) A method of treating or preventing obesity, comprising administering to a subject in need thereof an effective amount of a food or a beverage according to (10).

(18) A method of controlling weight gain, comprising administering to a subject in need thereof an effective amount of a food or a beverage according to (10).

(19) A method of effecting weight loss, comprising administering to a subject in need thereof an effective amount of a food or a beverage according to (10).

A composition, a food or a beverage containing a fermented soybean paste-like food material of the present invention has an anti-obesity action. In addition, a composition, a food or a beverage of the present invention is healthy in the light of its salt content since it contains a salt free or reduced salt fermented soybean paste-like food material as a key material. Further, since most of the proteins in said fermented soybean paste-like food material are hydrolyzed to amino acids, a composition, a food or a beverage containing said fermented soybean paste-like food material exhibits almost no bitterness.

### BRIEF DESCRIPTION OF THE DRAWINGS

A more complete appreciation of the invention and many of the attendant advantages thereof will be readily obtained as the same become better understood by reference to the following detailed description when considered in connection with the accompanying drawings, wherein:

FIG. 1 shows the weight of white adipose tissue per body weight of rats when the invention sample (dark bar) or control sample (open bar) was fed (the values are means±SEM); and

FIG. 2 shows the weight of brown adipose tissues per body weight of rats when the invention sample (dark bar) or control sample (open bar) was fed (the values are means±SEM, and *: Significantly different from control group (p<0.05)).

### DETAILED DESCRIPTION OF THE PREFERRED EMBODIMENTS

The present invention is described in detail as follows. When a fermented soybean paste (miso) is produced from soybean, salt is usually used to prevent proliferation of spoilage bacteria. The amount of salt added in a traditional fermented soybean paste (miso) is more than 10% of the weight of the unrefined soy, and the amount of salt added in a reduced salt fermented soybean paste (miso) is 5-10% of the weight of the unrefined soy at most. In the present invention, the % indication means “weight %” unless it is specified differently.

In the present invention, the soybean needs to be hydrolyzed under a salt free or a low salt condition. In case of a hydrolyzation under a low salt condition, the amount of salt added in the unrefined soy is 5% or less of the weight of the unrefined soy, preferably 1% or less, and more preferably 0.1% or less.

Therefore, the salt content in the fermented soybean paste-like food material is 5% or less, preferably 1% or less, and more preferably 0.1% or less, by weight based on the weight of the unrefined soy.

In order to prevent spoilage bacteria from proliferating during the hydrolyzation process, bacteriocin produced by lactic acid bacteria is substituted for salt in the present invention. The fermented soybean paste-like food material in the present invention may be prepared in the following manner. In the following description, the term “TEIRE”-treatment means a step of stirring the koji mixture in the koji making machine regularly for the purpose of accelerating the growth of the Koji mold.

(i) Step 1: adding a koji mold and a culture broth of a bacteriocin producing lactic acid bacterium, or a supernatant thereof, to steam-cooked soybean or a steam-cooked mixture of soybean and sub-material (e.g., rice and/or wheat);

(ii) Step 2: making koji from the mixture in (i) in a koji-making machine in which the outside air is excluded, with the exception of a time period of “TEIRE”-treatment, while supplying sterilized air continuously or intermittently;

(iii) Step 3: mixing the resulting koji with a culture broth of a bacteriocin producing lactic acid bacterium, or a supernatant thereof, to form a koji and culture broth mixture;

Optional step: mixing the koji and culture broth mixture with a food material selected from the group consisting of rice, wheat, soybean, and mixtures thereof in an amount of 0.01 to 50 times by weight of the resulting koji to form a koji, culture broth, and food material mixture;

(iv) Step 4: forming an unrefined soy by making the paste form of the koji, culture broth (and food material) mixture; and

(iv) Step 5: hydrolyzing the unrefined soy in a salt free or a low salt condition where the salt content of the unrefined soy is 5% by weight, based on the total weight of the hydrolyzed unrefined soy, or less

The main plant material used for the koji preparation in the present invention is soybean. Soybean may be used solely or rice and/or wheat may be added to soybean if necessary. Such raw materials may be subjected to a treatment of soaking in water, and a treatment of steam-cooking as needed.

A culture broth of a bacteriocin producing lactic acid bacterium or a supernatant thereof in the present invention is not limited as long as it can prevent spoilage bacteria from proliferating. The bacteriocin produced by lactic acid bacteria include nisin, pediocin, sakacin, nukacin, and the like. Among them, nisin is preferably used in light of the wide antibacterial spectrum. Nisin A, nisin Z and analogues thereof are reported as nisin produced by lactic acid bacteria. In the present invention, any nisin may be used.

Higher bacteriocin activity in the culture broth of the lactic acid bacterium or a supernatant thereof for use is desired for preventing contamination with and proliferation of microorganisms. When nisin is used, the nisin content in the culture broth of a lactic acid bacterium or the supernatant thereof needs to be 1000 IU/ml or more, preferably 3000 IU/ml or more.

Known lactic acid bacteria producing nisin may be used. For example, L. lactis AJ110212 (FERM BP-8552) and the like may be used. L. lactis AJ110212 strain was deposited on Nov. 19, 2003 with National Institute of Advanced Industrial Science and Technology, Patent Microorganisms Depositary Center under the accession number of FERM BP-8552.

According to the present invention, the aseptic airtight container for use is not particularly limited as long as production of the solid koji and hydrolysis of the unrefined soy of fermented soybean paste-like food material can be aseptically conducted while maintaining the bacteriostatic state. It is desired that one having the capability of supplying sterilized air into the koji making machine and having a structure capable of shutting off the interior of the koji making machine from the ambient air, for example, a koji making machine of the rotary drum type is used.

In the present invention, the Koji mold is not particularly limited as long as it is any one used in traditional fermented soybean paste. For example, Asperugillus oryase or Aspergillus sojae can be used. Alternatively, A. oryase and A. sojae can be used in combination. According to the action of Koji mold, raw material proteins are hydrolyzed to amino acids. As the result, the fermented soybean paste-like food material exhibit strong umami taste, richness, and thickness.

The point of the present invention is to use the fermented soybean paste-like food material produced by the afore-mentioned method as an anti-obesity composition. The anti-obesity composition in the invention may contain said fermented soybean paste-like food material in an amount of 0.1% -100% by weight, based on the total weight of the composition, or preferably 1-90% by weight, based on the total weight of the composition. Said anti-obesity composition is usually distributed in a form of a supplement, a food, or a beverage. The composition may be prepared by mixing said fermented soybean paste-like food material with other edible food materials, such as animal proteins, plant proteins, salt, seasonings, dextrin, sugars, and so on. An indication that the composition can be used for improving an anti-obesity action may be labeled on its product package.

A food or a beverage having an anti-obesity action can be prepared by mixing said fermented soybean paste-like food material with other edible food materials. Other food materials are not limited as long as they are edible and/or processable.

For example, sauces, salt, vinegars, seasonings, spices, fermented soybean paste, grains, potatoes, starches, sugars, sweeteners like aspartame, beans, fruits, vegetables, mushrooms, algae, seafood, meats (including meat products such as gelatin), eggs, dairy foods (milk, yogurt, etc), oils, luxury beverages like carbonated beverages, and the like can be used.

In the present invention, the mixing ratio of the fermented soybean paste-like food material and other food materials is not limited. The fermented soybean paste-like food material may be mixed in an amount of 0.1-99 parts by weight, or preferably 1-90 parts by weight, per 100 parts by weight of a food or a beverage.

A food or a beverage obtained according to the invention can be distributed in general form. It may be in paste, powder, liquid, or solid form. Illustrative examples of the process for powderization include vacuum drying processes, freeze-drying processes, spray dry processes, drum drier processes, vacuum drum dryer processes, micro wave drying processes, and the like.

Solid shaped foods are also included in the present invention. For example, bar-shaped foods, baked confectioneries, or wet type foods, containing 10 to 95% by weight of moisture, are included. The bar-shaped food may be prepared by shaping in e.g., a columnar shape, and may be either a wet type prepared by packing into a mold, or a dry type prepared by baking. Examples of the baked confectionery include cookies, biscuits, muffins, sponge cakes, rice crackers, and the like. Wet type foods referred to herein mean solid or semisolid foods containing 10 to 95% by weight of moisture such as cheese, bean curd (tofu), yogurt, pudding, and the like. Examples of the process for shaping include packing processes in a mold, hardening processes with an enzyme, hardening processes with dehydration, and heating-denaturing through application of heat such as baking, steaming and the like, hardening processes with an oil or a fat, hardening processes with gelatin, a polysaccharide or a thickener, hardening processes with a coagulating agent, hardening processes with freezing, hardening processes with drying, and the like.

In the present invention, the enzyme for use upon shaping is not particularly limited as long as it enables shaping through mixing with the fermented soybean paste-like food material and the other foodstuff. For example, transglutaminase can be illustrated.

In the present invention, when the process for shaping is a method in which baking is conducted, baking with an oven or the like may be carried out after mixing the fermented soybean paste-like food material and a food material other than the fermented soybean paste-like food material, and the process for baking is not particularly limited. The baking temperature is not particularly limited as long as it falls within a range that allows shaping the food. Shaping can be controlled by extending the baking time period when the temperature is low. To the contrary, when the temperature is high, it can be controlled by shortening the baking time period. In general, the temperature employed in the baking step, i.e., 90 to 230° C. is preferably since ordinary baking equipment can be used. In light of the operation, the temperature of 100 to 180° C. is more preferred.

The food or a beverage having an anti-obesity action in the invention may be in liquid form or a jelly.

An example of a preferable food or beverage includes a seasoning, a soup, a sauce, a dressing, jam, a cereal, bread, confectionery, a luxury beverage, or a cooked processed food.

The composition, the food or the beverage having an anti-obesity action in the invention is a salt free or a low salt product. In addition, it contains a lot of bioactive substances such as isoflavone and saponin. An indication that the food or the beverage can be used for improving an anti-obesity action may be labeled on its product package.

The food material, food, or beverage of the present invention may be used to treat or prevent obesity in a human or animal in need thereof. In particular, the food material, food, or beverage may replace part of the subject's dietary intake. Administration of a food material, food, or beverage of the present invention may be used by itself to treat or prevent obesity. Alternatively, administration of a food material, food, or beverage of the present invention may be used in combination with an exercise program and/or a reduced calorie diet to treat or prevent obesity. The food material, food, and beverage of the present invention may likewise be used to control weight gain and/or effect weight loss in a human or animal in need thereof.

Other features of the invention will become apparent in the course of the following descriptions of exemplary embodiments which are given for illustration of the invention and are not intended to be limiting thereof.

### EXAMPLES

### Example 1

Soybean in an amount of 50 kg was soaked in water to allow water absorption, followed by steam-cooking at 114° C. for 40 minutes in a steam cooker. The steam-cooked soybean was put in an airtight type koji making machine, and further mixed with 1 kg of a culture broth of L. lactis AJ110212 (FERM BP-8552) (nisin activity: 4500 IU/ml) and 0.05 kg of a koji mold starter (A. orizae, manufactured by Bioc), followed by subjecting to koji making at 30° C. for 43 hrs. For heat removal of the heat generated upon fermentation during koji making, a supply of the air sterilized by passing through a HEPA filter was conducted. During the koji making, koji was made in a koji making machine in which the outside air was shut out except for the time period of “TEIRE”-treatment (a step of stirring the mixture in the koji making machine for the purpose of accelerating the growth of the Koji mold). The “TEIRE”-treatment was carried out for 15 minutes every 10 hours after initiation of the koji making. To the thus-resulting solid koji was added 15.4 kg of a culture broth of L. lactis AJ110212 (FERM BP-8552) (nisin activity: 4500 IU/ml). After mixing, the mixture was subjected to grinding with a chopper to give a paste form. This paste was packed in a laminated pouch such that 500 g was filled in one pack. The unrefined soy of fermented soybean paste was hydrolyzed by incubating this pouch at 30° C. for 7 days, and a fermented soybean paste-like food material was obtained by heating at 80° C. for 40 minutes. To 8 kg of the fermented soybean paste-like food material were added a proper amount of water and 2.9 kg of dextrin as a bulking agent and mixed in a kneader.

The mixture was dried by a vacuum drum drier to 6 kg of the composition containing the fermented soybean paste-like food material of the invention. The fermented soybean paste-like food material was prepared in the absence of salt.

### Example 2

The composition prepared by the aforementioned vacuum drum drying was mixed to prepare a feed, wherein the amount of the composition added was 70% by weight based on the total weight of the feed. The prepared feed was fed to mice. Subsequently, it was found that the long term feeding of the composition of the invention suppressed the increase of body weight of mice. As a control, a non-fermented soybean material prepared by the following method was used. Soybean in an amount of 7 kg was soaked in water to allow water absorption, followed by steam-cooking at 114° C. for 40 minutes in a steam cooker. Then, the steam-cooked soybean was subjected to grinding with a chopper to give a paste form. To 8 kg of the paste were added a proper amount of water and 2.9 kg of dextrin as a bulking agent and mixed in a kneader. The mixture was dried by a vacuum drum drier to around 6 kg of the composition containing the non-fermented soybean material (the control).

The detailed test method is described as follows. 18 male mice of B6.129P2-Apoe<TM1UNC>/J of 8 weeks age were used and divided into two groups so that there was no difference in weight. The feed containing the composition of the invention was freely (not compulsorily) fed to one group for 3 months and the feed containing the non-fermented soybean material (the control) was freely (not compulsorily) fed to the other group for 3 months. The fat content in each feed was 20% and the total nitrogen content in each feed was the same. Further, no substance having an anti-obesity action such as capsaicin was added in any of the feed.

Subsequently, the body weight of the mice of the test group decreased significantly compared to that of the control group (Table 1). From this result, it was found that the composition of the present invention exhibits an action of suppression of a body weight increase in comparison with the composition containing the non-fermented soybean material (the control). And, it was indicated that the composition of the invention has an anti-obesity action in mice.

TABLE 1

| | Control Group | Test Group | Statistical Significance |
|---|---|---|---|
| Before the test | 23.2 ± 0.57 g | 23.1 ± 0.60 g | not significant |
| After the 90 | 28.3 ± 0.57 g | 25.1 ± 0.53 g | p < 0.01 |
| days test | | | |

Values are means ± SEM (n = 9)

### Example 3

The composition prepared by the method described in Example 1 was mixed to prepare a feed, wherein the amount of the composition added was 70% by weight based on the total weight of the feed. The prepared feed was fed to rats.

Subsequently, it was found that the long term feeding of the composition of the invention suppress increases of body weight of rats. As a control, a non-fermented soybean material prepared by the method described in Example 2 was used.

The detailed test method is described as follows. 20 male rats of Wistar strain of 5 weeks age were used and divided into two groups so that there was no difference in weight. The feed containing the composition of the invention was fed to one group for 2 weeks, and the feed containing the non-fermented soybean material (the control) was fed to the other group for 2 weeks. The fat content in each feed was 20% and the total nitrogen content in each feed was the same. Further, no substance having an anti-obesity action such as capsaicin was added in any of the feed. After the feeding test was completed, the rats were submitted to dissection and the white adipose tissues and the brown adipose tissues were weighed.

Subsequently, the body weight of the rats of the test group decreased significantly as compared to that of the control group (Table 2). The amount of the increase of the body weight per the intake of the feeds was calculated as the feed efficiency and the value of the test group was significantly lower (Table 2). From the results, it was indicated that the composition of the present invention exhibits an action of suppression of a body weight increase in comparison with the composition containing the non-fermented soybean material (the control).

TABLE 2

| | Control Group | Test Group | Statistical Significance |
|---|---|---|---|
| Before the test | 181.8 ± 2.6 g | 180.0 ± 1.8 g | not significant |
| After the 14 | 242.4 ± 4.2 g | 213.4 ± 2.2 g | p < 0.01 |
| days test | | | |
| Feed Efficiency | 0.229 ± 0.007 | 0.137 ± 0.008 | p < 0.01 |

Values are means ± SEM (n = 10)

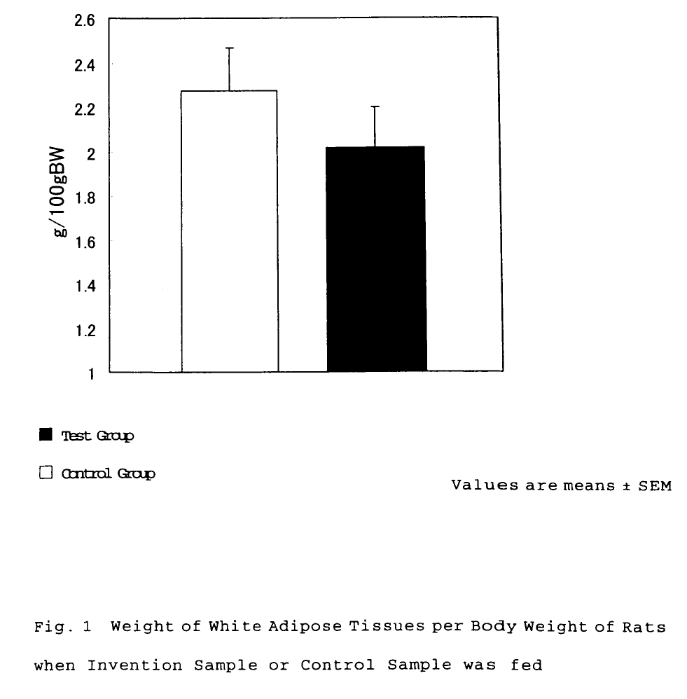

Further, it was found that the weight of the white adipose tissues (the total amount of the weight of perirenal fats, epididymal fats, and retroperitoneal fats) per body weight of rats having taken the invention sample tends to be lighter compared to that of the control group (see, FIG. 1). According to the result, it was indicated that the composition of the invention exhibits an action of suppression of fat accumulation.

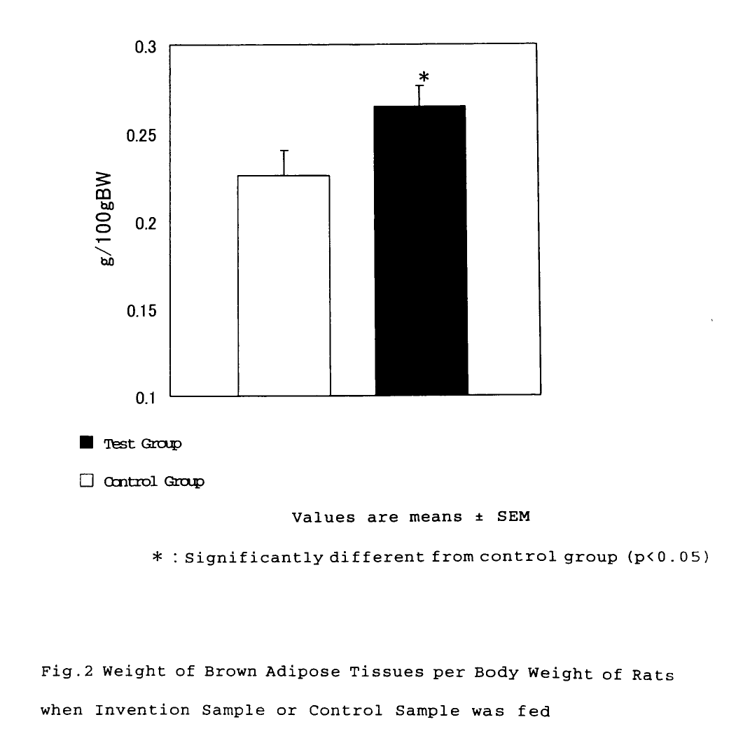

The weight of the brown adipose tissues between shoulder blades per body weight of the rats of the test group was significantly heavier compared to that of the control group (see, FIG. 2). It is reported that brown adipose tissues contains mitochondria in plenty and accelerates energy consumption through a heat generation. Therefore, it was indicated that the composition of the invention possibly accelerates energy consumption in comparison with the composition containing the non-fermented soybean.

It was indicated in rats that the composition of the invention exhibits anti-obesity actions such as suppression of body weight increase, suppression of fat accumulation, acceleration of energy consumption and so on.

### INDUSTRIAL APPLICABILITY.

A composition, a food or a beverage containing the fermented soybean-like food material is useful and valuable since it exhibits an anti-obesity action.

Obviously, numerous modifications and variations of the present invention are possible in light of the above teachings. It is therefore to be understood that, within the scope of the appended claims, the invention may be practiced otherwise than as specifically described herein.

All patents and other references mentioned above are incorporated in full herein by this reference, the same as if set forth at length.

1. A method of controlling weight gain, comprising administering to a subject in need thereof an effective amount of a salt-free or a reduced-salt fermented soybean paste-like food material, which is obtained by hydrolyzing soybean or a mixture of soybean and a sub-material with a koji mold, and wherein the salt content in the fermented soybean paste-like food material is 5% by weight, based on the total weight of said food material, or less.

2. A method according to claim 1, wherein said sub-material is at least one member selected from the group consisting of rice, wheat, and mixtures thereof.

3. A method according to claim 1, wherein said koji mold is at least one member selected from the group consisting of Aspergillus sojae, Aspergillus oryzae, and a mixture thereof.

4. A method according to claim 1, wherein said fermented soybean paste-like food material is prepared by a process comprising:
(i) adding (a) a koji mold and (b) a culture broth of a bacteriocin producing lactic acid bacterium or a supernatant of a culture broth of a bacteriocin producing lactic acid bacterium which comprises said bacteriocin, to (c) soybean or a mixture of soybean and a sub-material, to obtain a first mixture;
(ii) making koji from said first mixture in a koji-making machine in which the outside air is excluded, with the exception of a time period of “TEIRE”-treatment, while supplying sterilized air continuously or intermittently, to obtain koji;
(iii) mixing said koji with a culture broth of a bacteriocin producing lactic acid bacterium or a supernatant of a culture broth of a bacteriocin producing lactic acid bacterium which comprises said bacteriocin, to obtain a koji and culture broth mixture;
(iv) forming a paste of said koji and culture broth mixture, to obtain an unrefined soy; and
(v) hydrolyzing said unrefined soy in a low salt condition, to obtain said food material which has a salt content of 5% by weight, based on the total weight of said food material, or less.

5. A method according to claim 4, wherein said process comprises:
(iii′) after said mixing said koji with said culture broth of a bacteriocin producing lactic acid bacterium or said supernatant of a culture broth of a bacteriocin producing lactic acid bacterium which comprises said bacteriocin, to obtain said koji and culture broth mixture, additionally mixing said koji and culture broth mixture with a food material selected from the group consisting of rice, wheat, soybean, and mixtures thereof in an amount of 0.01 to 50 times by weight of said koji to form a koji, culture broth, and food material mixture, and
(iv′) making a paste of said koji, culture broth, and food material mixture, to obtain said unrefined soy.

6. A method according to claim 4, wherein said sub-material is at least one member selected from the group consisting of rice, wheat, and mixtures thereof.

7. A method according to claim 4, wherein said bacteriocin is nisin.

8. A method according to claim 7, wherein the nisin content in said culture broth of a lactic acid bacterium or said supernatant of a culture broth of a bacteriocin producing lactic acid bacterium which comprises said bacteriocin is 1000 IU/ml or more.

9. A method according to claim 4, wherein said lactic acid bacterium is Lactococcus lactis.

10. A method according to claim 1, wherein said food material is contained in a package and an indication that said food material has an anti-obesity action is labeled on said package.

11. A method of treating or preventing obesity, comprising administering to a subject in need thereof an effective amount of a salt-free or a reduced-salt fermented soybean paste-like food material, which is obtained by hydrolyzing soybean or a mixture of soybean and a sub-material with a koji mold, and wherein the salt content in the fermented soybean paste-like food material is 5% by weight, based on the total weight of said food material, or less.

12. A method according to claim 11, wherein said sub-material is at least one member selected from the group consisting of rice, wheat, and mixtures thereof.

13. A method according to claim 11, wherein said koji mold is at least one member selected from the group consisting of Aspergillus sojae, Aspergillus oryzae, and a mixture thereof.

14. A method according to claim 11, wherein said fermented soybean paste-like food material is prepared by a process comprising:
(i) adding (a) a koji mold and (b) a culture broth of a bacteriocin producing lactic acid bacterium or a supernatant of a culture broth of a bacteriocin producing lactic acid bacterium which comprises said bacteriocin, to (c) soybean or a mixture of soybean and a sub-material, to obtain a first mixture;
(ii) making koji from said first mixture in a koji-making machine in which the outside air is excluded, with the exception of a time period of “TEIRE”-treatment, while supplying sterilized air continuously or intermittently, to obtain koji;
(iii) mixing said koji with a culture broth of a bacteriocin producing lactic acid bacterium or a supernatant of a culture broth of a bacteriocin producing lactic acid bacterium which comprises said bacteriocin, to obtain a koji and culture broth mixture;
(iv) forming a paste of said koji and culture broth mixture, to obtain an unrefined soy; and
(v) hydrolyzing said unrefined soy in a low salt condition, to obtain said food material which has a salt content of 5% by weight, based on the total weight of said food material, or less.

15. A method according to claim 14, wherein said process comprises:
(iii′) after said mixing said koji with said culture broth of a bacteriocin producing lactic acid bacterium or said supernatant of a culture broth of a bacteriocin producing lactic acid bacterium which comprises said bacteriocin, to obtain said koji and culture broth mixture, additionally mixing said koji and culture broth mixture with a food material selected from the group consisting of rice, wheat, soybean, and mixtures thereof in an amount of 0.01 to 50 times by weight of said koji to form a koji, culture broth, and food material mixture, and
(iv′) making a paste of said koji, culture broth, and food material mixture, to obtain said unrefined soy.

16. A method according to claim 14, wherein said sub-material is at least one member selected from the group consisting of rice, wheat, and mixtures thereof.

17. A method according to claim 14, wherein said bacteriocin is nisin.

18. A method according to claim 17, wherein the nisin content in said culture broth of a lactic acid bacterium or said supernatant of a culture broth of a bacteriocin producing lactic acid bacterium which comprises said bacteriocin is 1000 IU/ml or more.

19. A method according to claim 14, wherein said lactic acid bacterium is Lactococcus lactis.

20. A method according to claim 11, wherein said food material is contained in a package and an indication that said food material has an anti-obesity action is labeled on said package.

21. A method of effecting weight loss, comprising administering to a subject in need thereof an effective amount of a salt-free or a reduced-salt fermented soybean paste-like food material, which is obtained by hydrolyzing soybean or a mixture of soybean and a sub-material with a koji mold, and wherein the salt content in the fermented soybean paste-like food material is 5% by weight, based on the total weight of said food material, or less.

22. A method according to claim 21, wherein said sub-material is at least one member selected from the group consisting of rice, wheat, and mixtures thereof.

23. A method according to claim 21, wherein said koji mold is at least one member selected from the group consisting of Aspergillus sojae, Aspergillus oryzae, and a mixture thereof.

24. A method according to claim 21, wherein said fermented soybean paste-like food material is prepared by a process comprising:
(i) adding (a) a koji mold and (b) a culture broth of a bacteriocin producing lactic acid bacterium or a supernatant of a culture broth of a bacteriocin producing lactic acid bacterium which comprises said bacteriocin, to (c) soybean or a mixture of soybean and a sub-material, to obtain a first mixture;
(ii) making koji from said first mixture in a koji-making machine in which the outside air is excluded, with the exception of a time period of “TEIRE”-treatment, while supplying sterilized air continuously or intermittently, to obtain koji;
(iii) mixing said koji with a culture broth of a bacteriocin producing lactic acid bacterium or a supernatant of a culture broth of a bacteriocin producing lactic acid bacterium which comprises said bacteriocin, to obtain a koji and culture broth mixture;
(iv) forming a paste of said koji and culture broth mixture, to obtain an unrefined soy; and
(v) hydrolyzing said unrefined soy in a low salt condition, to obtain said food material which has a salt content of 5% by weight, based on the total weight of said food material, or less.

25. A method according to claim 24, wherein said process comprises:
(iii′) after said mixing said koji with said culture broth of a bacteriocin producing lactic acid bacterium or said supernatant of a culture broth of a bacteriocin producing lactic acid bacterium which comprises said bacteriocin, to obtain said koji and culture broth mixture, additionally mixing said koji and culture broth mixture with a food material selected from the group consisting of rice, wheat, soybean, and mixtures thereof in an amount of 0.01 to 50 times by weight of said koji to form a koji, culture broth, and food material mixture, and
(iv′) making a paste of said koji, culture broth, and food material mixture, to obtain said unrefined soy.

26. A method according to claim 24, wherein said sub-material is at least one member selected from the group consisting of rice, wheat, and mixtures thereof.

27. A method according to claim 24, wherein said bacteriocin is nisin.

28. A method according to claim 27, wherein the nisin content in said culture broth of a lactic acid bacterium or said supernatant of a culture broth of a bacteriocin producing lactic acid bacterium which comprises said bacteriocin is 1000 IU/ml or more.

29. A method according to claim 24, wherein said lactic acid bacterium is Lactococcus lactis.

30. A method according to claim 21, wherein said food material is contained in a package and an indication that said food material has an anti-obesity action is labeled on said package.
